(19) United States
(12) Patent Application Publication
(10) Pub. No.: US 2004/0248864 A1
(43) Pub. Date: Dec. 9, 2004

(54) Use of anthroquinones in the treatment of kidney disease

(76) Inventors: Robin Mark Bannister, Essex (GB); Alan Rothaul, Essex (GB); Nicola Cooper, Essex (GB)

Correspondence Address: SALIWANCHIK LLOYD & SALIWANCHIK, A PROFESSIONAL ASSOCIATION, PO BOX 142950, GAINESVILLE, FL, 32614-2950, US

(21) Appl. No.: 10/487,737

(22) PCT Filed: Aug. 27, 2002
(86) PCT No.: PCT/GB02/03921

(30) Foreign Application Priority Data
Aug. 25, 2001 (GB) 0120788.5

(51) Int. Cl. A61K 31/56; A61K 31/12

(52) U.S. Cl. 514/171; 514/680

(57) ABSTRACT

An anthroquinone such as diacerein is useful for the treatment of renal conditions and conditions (such as SLE) which lead to renal damage.

### FIELD OF THE INVENTION

This invention relates to the use of anthroquinones in the treatment of kidney disease.

### BACKGROUND OF THE INVENTION

Kidney diseases include serious, life-threatening conditions such as nephritis, nephrosis and nephritic syndrome. Current treatments of these conditions utilise corticosteroids, antibiotics and immunosuppressants. However, these treatments are often associated with severe side-effects, e.g. steroid toxicity. In a significant proportion of patients, these treatment regimes are not effective, many patients requiring dialysis and transplantation.

A related condition, i.e. lupus erythematosus, is a serious life-threatening autoimmune disease. Systemic lupus erythematosus (SLE) typically leads to the development of nephritis and degeneration of the kidney. Consequent renal impairment is a serious complication of itself, leading to death. Currently, this aspect of lupus is poorly treated; while steroids, cytotoxic agents and immunosuppressants are used, these agents in themselves are toxic and, in a significant proportion of patients, renal transplantation is the only option.

Diacerein and an active metabolite, rhein, have been shown to reduce the production of the pro-inflammatory cytokine IL-1; see Yaron et al, Osteoarthritis and Cartilage 1999, 7(3): 272-280; and Moldovan, Osteoarthritis and Cartilage 2000, 8(3): 186-196. In addition, it has been reported that diacerein and rhein reduce the production of inducible nitric oxide synthase and production of nitric oxide (osteochondrocytes-IL-1b stimulated); see Pelletier, J. Rheumatology (1998).

Recently, it has been established that rhein down-regulates the production of key matrix metalloproteinases MMPs 1, 3, 9 & 13 and pro-MMPs, and additionally increases the production of a key natural inhibitor of MMP's, i.e. tissue inhibitor of matrix MMP-1, TIMP-1. The overall result of this activity is a reduction of proteolytic activity; see Tamura, et al, Osteoarthritis and Cartilage, 2001, 9:257-263.

### SUMMARY OF THE INVENTION

The present invention is based on the discovery that diacerein, e.g. in combination with pre-existing therapies, is useful for the treatment of renal disease, including renal impairment associated with lupus erythematosus. The efficacy of existing treatment, with corticosteroids, antibiotics or immunosuppressants, may be enhanced. In addition, the side-effects of the existing therapies may be ameliorated by reduction of dose.

According to the present invention, a compound selected from rhein and derivatives thereof is used for the manufacture of a medicament for the treatment of a renal condition or a condition which leads to renal damage.

### BRIEF DESCRIPTION OF THE DRAWINGS

FIG. 1 is a PK profile, i.e. a graph of plasma concentration of rhein (mg/ml) against time (min) post-dosing with 100, 200 and 300 mg/kg diacerein.

FIG. 2 is a diagram showing proteinurea (mg protein/day) with respect to time (days post-insult) for 5 treatments, respectively control, 2 mg/kg prednisolone, and 50, 100 and 200 mg/kg diacerein.

FIG. 3 is a diagram showing total crescent count score for the same 5 treatments as reported in FIG. 2.

### DESCRIPTION OF PREFERRED EMBODIMENTS

Conditions that can be treated according to the invention are indicated above, and include nephritis, nephrosis, nephrotic syndrome, lupus erythematosus and also SLE.

Compounds that can be used in the invention are anthroquinone derivatives of the formula

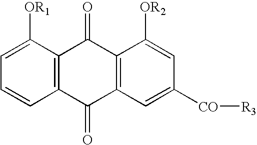

wherein R₁═R₂═H and R₃═OH (rhein) or R₁═R₂═Ac and R₃═OH (diacerein), as well as monoacetylrhein, or a prodrug of any of these active components. Such prodrugs include esters,. amides and salts, e.g., acyl derivatives at R₁ and R₂, and ester and amide derivatives at R₃. Compounds of this type, and their preparation, are described in, inter alia, U.S. Pat. No. 6,057,461 and EP-A-0570091, the contents of which are incorporated herein by reference.

For use, the active agent is typically formulated, e.g. with a conventional diluent or carrier, in a medicament adapted to be delivered by the oral, intravenous, rectal, vaginal, topical to skin, inhalation or intraarticular route. Oral delivery is preferred. Such formulations and suitable dosages are known to those skilled in the art, and will be chosen according to the usual considerations such as the potency of the drug, the severity of the condition and the route of administration.

Suitable compositions for oral use include tablets, troches, lozenges, aqueous or oily suspensions, dispersible powders or granules, emulsions, hard or soft capsules, syrups and elixirs. Suitable additives include sweetening agents, flavoring agents, coloring agents and preserving agents. Tablets contain the active ingredient in admixture with non-toxic pharmaceutically acceptable excipients, e.g. inert diluents such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastointestinal tract and thereby provide a sustained action over a longer period. For example, a time delay material such as glyceryl monostearate or glyceryl distearate may be employed. They may also be coated, to form osmotic therapeutic tablets for controlled release. Hard gelatin capsules may include an inert solid diluent, for example calcium carbonate, calcium phosphate or kaolin; soft gelatin capsules may include water or an oil medium, for example peanut oil, liquid paraffin or olive oil.

The drug may be delivered in combination with other therapies that are used to alleviate the symptoms of kidney disease. Drugs known for this purpose include corticosteroids, cytotoxic agents, immunosuppressants (e.g. azathioprine or 6-mercaptopurine) and antibiotics (e.g. metronidazole, ciprofloxacin or augmentin). It may also be used in combination with any drug that has prostanoid effects. Use of the drug in combination therapy may allow reduction of toxic immunosuppressants (e.g. steroid sparing).

The following study provides evidence on which the invention is based. This study shows that diacerein had a significant effect on both proteinuria and renal damage associated with nephrotoxic nephritis. The effect was even better than that seen for prednisolone which was overwhelmed by nephrotoxic nephritis at the end of the experiment. Considering that prednisolone is a steroid (one of the select drugs with sufficient anti-inflammatory action to be effective clinically), this is potentially an important clinical result. This is particularly true in view of the fact that diacerein has far fewer and less severe side-effects than prednisolone and related anti-nephritis drugs such as cyclophosphamide and azathioprine.

Study

Experiments were conducted, using a rat model of nephritis (nephrotoxic nephritis) (Karkar et al, Kidney International, 1997). This is an aggressive immune driven model which parallels clinical conditions such as lupus nephritis, glomerulonephritis, IgA nephropathy and other immune-mediated nephritic diseases.

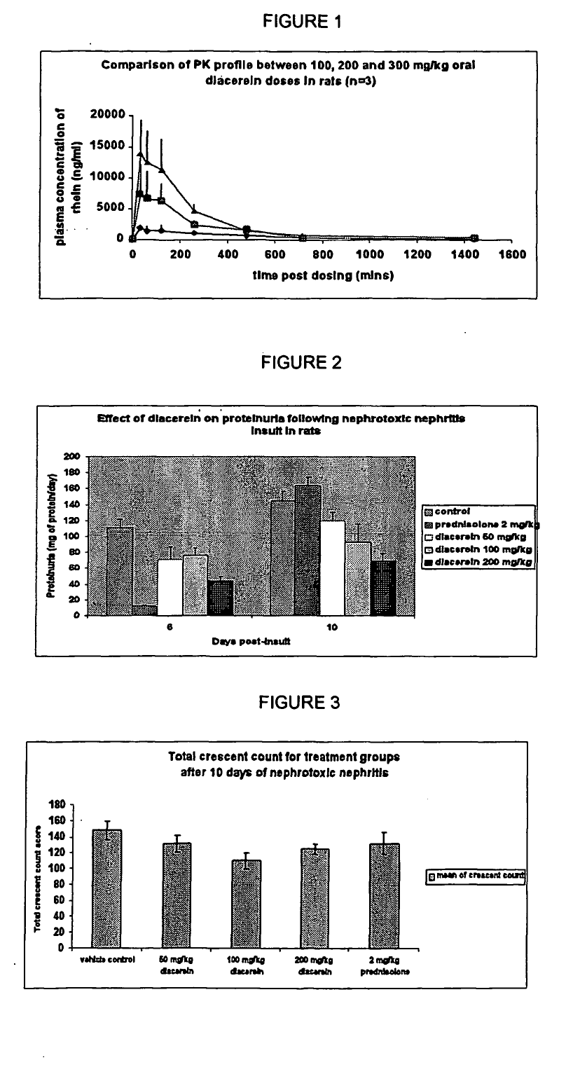

Firstly, the pharmacokinetic parameters were determined; this is shown in FIG. 1. It was found that the oral dose required to obtain therapeutic plasma concentrations of drug (Nicolas et al, Drug Disposition, 1998) was much higher in the rat than in humans (100-200 mg/kg compared to 0.8 mg/kg). Consequently, the dosing for the nephrotoxic nephritis experiment was based around this dose range (see below).

Diacerein was administered orallyonce a day throughout the experiment, at 50, 100 and 150 mg/kg of diacerein. Development of nephrotoxic nephritis was measured by the degree of proteinuria (in the control group) at days 6 and 10, post-disease initiation and by renal histology at day 10, quantified by the number of damaged nephrons (crescents) per histology section. These end-points were used to determine the success of the diacerein treatment. Alongside diacerein treatment, a negative control (drug vehicle only) and positive control (2 mg/kg prednisolone) were tested.

After diacerein treatment, proteinuria was found to be decreased compared to the control at day 6 in a dose-dependant manner, reaching statistical significance at the highest dose (*p<0.01). The effect was seen again at 10 days, with a dose-dependant decrease in proteinuria, compared to the control, reaching statistical significance at the top dose (*p<0.01). In the control group, proteinuria increased over time as anticipated, while prednisolone treatment markedly reduced proteinuria at day 6, with an unexpected rebound above the control proteinuria levels at day 10 (see FIG. 2).

Histology data show a decrease in nephron damage at the 50 and 100 mg/kg diacerein doses with a slight increase again at the 200 mg/kg dose (see FIG. 3). The 100 mg/kg dose effect was significant (*p<0.05). The effect of prednisolone on renal damage was negligible (same as the control sections).

1. (cancel).

2. The method, according to claim 9, wherein the compound is delivered by the oral route.

3. The method, according to claim 9, wherein the compound is selected from the group consisting of rhein, monoacetylrhein and diacerin.

4. The method, according to claim 3, wherein the compound is diacerein.

5. The method, according to claim 9, wherein the condition is selected from the group consisting of nephritis, nephrosis and nephritic syndrome.

6. The method, according to claim 9, wherein the condition is systemic lupus erythematosus.

7. The method, according to claim 9, wherein the subject of treatment is also undergoing treatment with at least one other agent selected from the group consisting of corticosteroids, antibiotics and immunosuppressants.

8. The method, according to claim 6, wherein the subject of treatment is also undergoing treatment with at least one other agent selected from the group consisting of steroids, cytotoxic agents and immunosuppressants.

9. A method for treating a renal condition, or a condition which results in renal damage, wherein said method comprises administering, to a subject in need of such treatment, a compound selected from the group consisting of rhein and derivatives thereof.
